(19) United States
(12) Patent Application Publication
(10) Pub. No.: US 2025/0360198 A1
(43) Pub. Date: Nov. 27, 2025

(54) mRNA-based HIV Vaccine For Acute and Latent Infections

(71) Applicants: Matthias Magoola, Kampala (UG); Sarfaraz K. Niazi, Deerfield, IL (US); Zafeer Ahmad, Wadsworth, IL (US)

(72) Inventors: Matthias Magoola, Kampala (UG); Sarfaraz K. Niazi, Deerfield, IL (US); Zafeer Ahmad, Wadsworth, IL (US)

(21) Appl. No.: 18/741,686

(22) Filed: Jun. 12, 2024

Related U.S. Application Data
(63) Continuation-in-part of application No. 18/674,840, filed on May 25, 2024.

(51) Int. Cl. A61K 39/21; A61K 39/00

(52) U.S. Cl. CPC A61K 39/21; A61K 2039/53; A61K 2039/575

(57) ABSTRACT

The Human Immunodeficiency Virus (HIV) infection and recurrent infection prevention mRNA vaccine comprising epitopes of HIV-1 and HIV-2 viruses and their corresponding Nef proteins.

### CROSS-REFERENCE TO RELATED APPLICATION

This application is a Continuation-In-Part of U.S. patent application Ser. No. 18/674,840, filed on May 25, 2024, entitled “mRNA-based HIV Vaccine for Acute and Latent Infections” and incorporated herein by reference in its entirety.

### SEQUENCE LISTING

The instant application contains a Sequence Listing, which has been submitted electronically in ASCII format and is hereby incorporated by reference in its entirety. Said ASCII text file, created on Jun. 5, 2024, is named 2024-0420CIP1_HIVNEF_Sequence Listing, and is 9 kb in size.

### BACKGROUND OF THE INVENTION

The spread of HIV poses significant health risks worldwide. Conventional vaccination strategies have had limited success in providing broad protection against these viruses due to their high mutation rates and various strains. Therefore, an improved vaccine is needed to induce a robust and broad immune response against multiple strains of HIV.

Nucleoside vaccines are a significant pivot from traditional approaches, as they embody the synthesis of vaccines using nucleoside-modified mRNA. This approach has risen to prominence following the success of mRNA COVID-19 vaccines, which utilize lipid nanoparticles for delivery. The inherent adaptability of nucleoside vaccines has sparked interest in their application against various pathogens, potentially allowing for the design of vaccines that could be rapidly updated in response to viral mutations. This nucleoside-modified mRNA technology heralds a new era in vaccine development, standing on the shoulders of prior art but pushing the boundaries into novel territories of immunization strategies.

This paradigm shift towards nucleoside-modified mRNA vaccines represents a significant leap forward in immunization technology, particularly in the combat against complex viruses like HIV. The modularity of mRNA vaccines-allowing for the plug-and-play insertion of genetic codes for specific antigens-facilitates a versatile platform swiftly adapted to address emerging strains and variants, a precious characteristic given the mutagenic nature of viruses like HIV.

### SUMMARY OF THE INVENTION

Although HIV-2 infection is less geographically dispersed than HIV-1, and the HIV-2 epidemic is primarily focused on West Africa, HIV-2 is not uncommon in the epidemic in Europe and India because of travel between West Africa and Europe or India. HIV-1 and HIV-2 genomes share about 60% homology in conserved genes such as gag and pol and 35-45% homology in the env genes. The core proteins of HIV-1 and HIV-2 display frequent cross-reactivity, whereas the envelope proteins are more type-specific.

The HIV-1 envelope glycoprotein gp160 (P03377·ENV_HVIBR) and HIV-2 (gp125 Q74432·Q74432_9HIV2) oligomerizes in the host endoplasmic reticulum into predominantly trimers. In the second step, gp160 transits in the host Golgi, where glycosylation is completed. The precursor is then proteolytically cleaved in the trans-Golgi and activated by cellular furin or furin-like proteases to produce gp120 and gp41. The surface protein gp120 attaches the virus to the host lymphoid cell by binding to the primary receptor CD4. This interaction induces a structural rearrangement, creating a high-affinity binding site for a chemokine coreceptor like CXCR4 and CCR5 that acts as a ligand for CD209/DC-SIGN and CLEC4M/DC-SIGNR, which are respectively found on dendritic cells (DCs), and on endothelial cells of liver sinusoids and lymph node sinuses. These interactions allow these cells to capture viral particles at mucosal surfaces and then transmit them to permissive cells. HIV subverts the migration properties of dendritic cells to gain access to CD4+ T-cells in lymph nodes. Virus transmission to permissive T-cells occurs either in trans (without DCs infection, through viral capture and transmission) or cis (following DCs productive infection, through the usual CD4-gp120 interaction), thereby inducing a robust infection. In trans infection, bound virions remain infectious over days, and it is proposed that they are not degraded but protected in non-lysosomal acidic organelles within the DCs close to the cell membrane, thus contributing to the viral infectious potential during DCs' migration from the periphery to the lymphoid tissues. On arrival at lymphoid tissues, intact virions recycle back to DCs' cell surface, transmitting the virus to CD4+ T-cells. Transmembrane protein gp41 acts as a class I viral fusion protein. Under the current model, the protein has at least 3 conformational states: pre-fusion native state, pre-hairpin intermediate state, and post-fusion hairpin state. During the fusion of viral and target intracellular membranes, the coiled coil regions (heptad repeats) assume a trimer-of-hairpins structure, positioning the fusion peptide near the C-terminal region of the ectodomain. The formation of this structure appears to drive apposition and subsequent fusion of viral and target cell membranes. Complete fusion occurs in host cell endosomes and is dynamin-dependent; however, some lipid transfer might occur in the plasma membrane. The virus undergoes clathrin-dependent internalization long before endosomal fusion, thus minimizing the surface exposure of conserved viral epitopes during fusion and reducing the efficacy of inhibitors targeting these epitopes. Membrane fusion leads to the delivery of the nucleocapsid into the cytoplasm.

The invention relates to the technical field of biotechnology, to a therapeutic HIV mRNA combination vaccine comprising a combination of multiple messenger RNA (mRNA) molecules or combined into a single molecule that expresses conserved and immunogenic epitopes of HIV gp160 proteins P03377 ENV_HVIBR and HIV-2 (gp125 Q74432·Q74432_9HIV2 formulated within a lipid nanoparticle (LNP) delivery system to generate higher serum antibody level and simultaneously stimulate organisms to generate TH1 and TH2 type cellular immune response against the HIV.

Capsid proteins play crucial roles in the HIV lifecycle, making them potential targets for vaccine development alongside surface proteins. The capsid p24 (Q9WMV8· Q9WMV8_9HIV1) protein is an HIV core protein comprising the virus's structural core. It's often used as a marker in viral load tests to measure the amount of HIV in a person's blood.

The reverse transcriptase (RT) is an enzyme involved in the replication of HIV. It's targeted by some antiretroviral drugs. Integrase (IN) is another HIV enzyme targeted by certain antiretroviral medications. It plays a role in integrating the viral DNA into the host cell's genome. The protease (PR) is an enzyme that helps process viral proteins into their functional forms. Inhibition of PR is another target for antiretroviral drugs.

While surface proteins, particularly the envelope protein, are highly variable and mutate frequently, making vaccine development challenging, capsid proteins are more conserved, potentially offering a more stable target for the immune system. A vaccine targeting capsid and surface proteins could elicit a broader immune response, increasing the chances of preventing viral replication even when the virus mutates. However, capsid proteins are internal, making them less accessible to antibodies than surface proteins, which presents significant challenges in vaccine design. Moreover, since the immune system predominantly targets external structures, inducing an effective response against internal proteins like capsids may require innovative approaches. The concern about off-target effects is valid when considering the inclusion of capsid protein antibodies in an HIV vaccine. Off-target effects refer to the unintended actions of antibodies (or any therapeutic agents) on proteins or cells other than their intended targets, which can lead to adverse effects or toxicity.

In the case of antibodies against the HIV capsid protein, off-target effects could occur if the antibodies bind to human proteins that are similar in structure to the HIV capsid protein or if they interfere with cellular processes that are crucial for normal cell function. This can happen due to the phenomenon known as molecular mimicry, where a part of the HIV capsid protein resembles a sequence or structural element of a host protein.

These unintended interactions can lead to various complications, including immune responses against the body's tissues (autoimmunity), inflammation, or other cellular dysfunctions. Therefore, when designing and testing capsid protein antibodies for inclusion in HIV vaccines, researchers must thoroughly evaluate their specificity, binding affinity, and potential for off-target effects.

The development of combination antiretroviral therapy (cART) has improved the outcome for individuals with human immunodeficiency virus (HIV). However, HIV can persist even during cART by establishing a latent reservoir in a subset of infected cells. Eliminating these infected cells requires a combination of therapeutic agents with different mechanisms of action. Researchers have recently proposed a two-pronged “shock and kill” strategy for eradication. The “shock” step involves treatment with latency reversal agents (LRAs) to reactivate latent reservoirs of HIV-infected cells. Reactivated infected cells could then be killed through the activity of anti-HIV cytotoxic T-cell lymphocytes (CTL) or via the cytopathic effect of the virus. However, CTL recognition and destruction of infected cells depend on the expression of host major histocompatibility complex I (MHC-I)-encoded proteins on the cell surface. The HIV-Nef protein promotes evasion from CTL recognition by downmodulating MHC-I HLA-A and-B. Nef disrupts MHC-I cell surface expression by binding to the cytoplasmic tail and stabilizing the interaction between MHC-I and clathrin adaptor protein 1 (AP-1). The formation of this complex results in the targeting of MHC-I to the lysosome for degradation.

Factors of infectivity and pathogenicity are required for optimal virus replication. The complex alters numerous pathways of T-lymphocyte function and down-regulates immunity surface molecules to evade host defense and increase viral infectivity. The complex also alters the functionality of other immunity cells, like dendritic cells, monocytes/macrophages, and NK cells.

In infected CD4+T-lymphocytes, down-regulate the surface MHC-I, mature MHC-II, CD4, CD28, CCR5, and CXCR4 molecules. Mediates internalization and degradation of host CD4 through the interaction with the cytoplasmic tail of CD4, the recruitment of AP-2 (clathrin adapter protein complex 2), internalization through clathrin-coated pits, and subsequent transport to endosomes and lysosomes for degradation. Diverts host MHC-I molecules to the trans-Golgi network-associated endosomal compartments by an endocytic pathway to finally target them for degradation. MHC-I down-regulation may involve AP-1 (clathrin adapter protein complex 1) or possibly Src family kinase-ZAP70/Syk-PI3K cascade recruited by PACS2. In consequence, infected cells are masked for immune recognition by cytotoxic T-lymphocytes. Decreasing the number of immune receptors also prevents reinfection by more HIV particles (superinfection). It down-regulates host SERINC3 and SERINC5, excluding these proteins from the viral particles. Virion infectivity is drastically higher when SERINC3 or SERINC5 are excluded from the viral envelope because these host antiviral proteins impair the membrane fusion event necessary for subsequent virion penetration.

It bypasses host T-cell signaling by inducing a transcriptional program nearly identical to anti-CD3 cell activation. Interaction with the TCR-zeta chain up-regulates the Fas ligand (FasL). It increases surface FasL molecules and decreases surface MHC-I molecules on infected CD4+ cells, sending attacking cytotoxic CD8+T-lymphocytes into apoptosis.

It plays a role in optimizing the host cell environment for viral replication without causing cell death by apoptosis, as it protects the infected cells from apoptosis to keep them alive until the next virus generation is ready to strike. Inhibits the Fas and TNER-mediated death signals by blocking MAP3K5/ASK1. It decreases the half-life of TP53, protecting the infected cell against p53-mediated apoptosis. It also inhibits the apoptotic signals regulated by the Bcl-2 family proteins by forming a Nef/PI3-kinase/PAK2 complex that activates PAK2 and induces phosphorylation of BAD.

Extracellular Nef protein targets CD4+T-lymphocytes for apoptosis by interacting with CXCR4 surface receptors.

HIV-1 lineages are divided into three main groups: M (for Major), O (for Outlier), and N (for New, or Non-M, Non-O). Most strains found worldwide belong to the group M. Group O seems to be endemic to and largely confined to Cameroon and neighboring countries in West Central Africa, where these viruses represent a small minority of HIV-1 strains. A limited number of isolates from Cameroonian persons represent group N. Group M is subdivided into 9 clades or subtypes (A to D, F to H, J, and K).

The human immunodeficiency virus (HIV)-encoded accessory protein Nef enhances pathogenicity by reducing major histocompatibility complex I (MHC-I) cell surface expression, protecting HIV-infected cells from immune recognition. Designing an effective HIV-1 Nef inhibitor to achieve a potential cure has challenged researchers for decades. Nef is a multifunctional protein that disrupts signal transduction pathways and intracellular protein trafficking. In addition, Nef influences host cell proteins through distinct surface interactions rather than enzymatic or biochemical activity. This invention adds antibodies against Nef, along with antibodies to remove HIV, both at the cell and humoral level.

The Nef protein, encoded by the Nef gene in HIV-1, HIV-2, and simian immunodeficiency viruses (SIV), plays a critical role in the pathogenicity and progression of HIV to AIDS, contradicting its initial classification as a “Negative Factor.” This multifunctional protein is instrumental in evading the host's immune defenses by downregulating CD4 receptors and MHC class I molecules on the surface of infected cells, thereby hindering the immune system's ability to detect and eliminate the virus. Furthermore, Nef enhances the infectivity and replication of HIV by manipulating various cellular mechanisms, including altering cell signaling pathways to create an environment that favors viral replication.

Nef's influence extends beyond viral replication and modulates host cell functions, particularly affecting T-cell activation and apoptosis. By interacting with cellular signaling pathways, Nef can induce T cells' premature activation or death, undermining the host's immune response and promoting persistent infection. Additionally, Nef alters the actin cytoskeleton and cell trafficking, increasing the motility of infected cells and thereby facilitating the widespread distribution of the virus throughout the host's body.

The multifaceted functions of Nef, encompassing immune evasion, enhancement of viral replication and infectivity, and manipulation of host cell biology, underscore its significance in HIV's lifecycle and pathogenicity. Its central role in the progression of HIV infection makes it a potential target for antiretroviral therapy; however, the complexity and variability of its functions and sequences among different HIV strains present substantial challenges for drug development.

The present invention combines the epitopes of Nef protein from HIV-1 and HIV-2 with surface proteins of HIV-1 and HIV-2.

### DETAILS OF THE INVENTION

To make the objects, technical solutions, and advantages of the embodiments of the present invention more apparent, the technical solutions in the embodiments of the present invention will be clearly and completely described below, but not all embodiments of the present invention. Thus, the following detailed description of the embodiments of the present invention is not intended to limit the scope of the invention, as claimed, but is merely representative of selected embodiments of the invention. All other embodiments, which can be derived by a person skilled in the art from the embodiments given herein without making any creative effort, shall fall within the protection scope of the present invention.

In summary, for HIV, a strong T cell response is crucial, mainly because of the virus's ability to evade B cell responses due to high mutation rates.

HIV is a lentivirus that primarily infects hosts and cells through bodily fluids or pregnancy communication. HIV infects cells essential to the immune system, such as CD4+ T cells, macrophages, and dendritic cells, ultimately causing cell death. When the rate and magnitude of cell death cause essential cell levels to fall below critical levels, it becomes increasingly more difficult for the host to mount an effective immune response, leading to acquired immunodeficiency syndrome (AIDS). Without treatment, the average survival time after infection with HIV is estimated to be 9 to 11 years. In terms of HIV vaccine development, the unique challenge has been the virus's extreme genetic diversity and propensity for rapid mutation. However, the versatility of nucleoside-modified mRNA vaccines opens the door to creating multivalent vaccines that can target multiple strains of HIV simultaneously. Furthermore, as these vaccines can be produced with remarkable speed, they offer a promising solution to react quickly to the evolution of the virus within the population.

In contrast, the quest for an effective HIV vaccine has been challenging due to the virus's ability to evade the immune system. Initial vaccine designs attempted to stimulate neutralizing antibodies against the virus's envelope proteins. The RV144 trial (10.1089/aid.2012.0103) underscored the necessity of inducing antibodies and cellular immune responses. This led to the exploration of broadly neutralizing antibodies (bnAbs), which some individuals naturally produce against HIV. Viral vector vaccines and nucleoside-modified mRNA platforms have been investigated, the latter being a cutting-edge approach where mRNA is used to encode HIV antigens, exploiting its ability to present these antigens in their native conformations. Both prophylactic and therapeutic strategies have been employed, with the latter seeking to control the infection in people already living with HIV.

The choice of MHC alleles for HIV infection epitope prediction considers several factors specific to the virus's immunology and the population affected by the disease. HIV mutates rapidly; therefore, identifying epitopes conserved across multiple strains can be crucial for effective vaccine design or therapeutic interventions. For HIV, CTL (Cytotoxic T Lymphocyte) responses are crucial, and these responses are mediated by peptides presented on MHC class I molecules. MHC class I alleles are associated with better control of HIV infection: HLA-B*57 and HLA-B*27. The alleles have been associated with slower progression to AIDS, suggesting that epitopes presented by these alleles could effectively elicit protective T-cell responses. HLA-B*35: This allele, in contrast, is often associated with rapid disease progression, and understanding the epitopes presented by this allele might help in understanding mechanisms of immune escape.

For HIV infection, understanding which MHC Class II alleles to focus on for epitope prediction is essential for designing effective therapies and vaccines. These alleles present antigens to CD4+T-helper cells, crucial for orchestrating the immune response, including producing antibodies and activating cytotoxic T cells. The selection of class II alleles would similarly focus on common alleles and those associated with HIV disease progression or control. Some MHC Class II alleles that have been noted in the context of HIV research include DRB1*01: This allele has been associated with slower disease progression in some studies; DRB1*03: This allele group has shown to be important in the context of HIV, though the associations can be different depending on the specific allele; DRB1*04: Some alleles in this group are also important in HIV infection and can be associated with differences in disease progression; DRB1*07: This allele has shown some protective effects in specific populations; DRB1*11: Associations with slower progression of disease have been seen with this allele in some studies; DRB1*13: This allele is also notable in some studies for its role in HIV infection; DRB1*15: This allele may have a role in influencing the immune response to HIV. DQB1 and DPB1 alleles are also part of the MHC Class II region and can present antigens to CD4+ T cells. Some specific alleles within these loci may also affect the immune response to HIV.

Linear epitopes are recognized by T cells. These epitopes are often internal hydrophobic amino acid sequences processed by macrophages and presented to T cells in the context of human leukocyte antigen (HLA) class I and II molecules. Processed epitopes containing 7 to 17 amino acids are presented to T lymphocytes by antigen-presenting cells. B cell epitopes from globular proteins range from 5 to 30 amino acids and are usually conformational. The length and flexibility of the epitope ensure high-affinity binding to B cell receptors or circulating antibodies.

TABLE 1
Epitopes of proteins

| No | Peptides | Description Epitopes |
|---|---|---|
| HIVNEF_seq_1 | KQSSGGDPEI | gp160 P03377 B-Cell |
| HIVNEF_seq_2 | TWFNSTWSTEGSNNTEGSD | Epitope |
| HIVNEF_seq_3 | LGVAPTKAKRRVVQREK | |
| HIVNEF_seq_4 | RVKEKYQHLW | gp160 P03377 MHC |
| | | Class-1 T-cell epitope |
| HIVNEF_seq_5 | WNATLKQIASKLREQ | gp160 P03377 MHC |
| HIVNEF_seq_6 | KLDIIPIDNDTTSYT | Class-2 T-cell epitope |
| HIVNEF_seq_7 | GYFPDWQNYTPGPGIRY | NEF P05856 B-Cell |
| HIVNEF_seq_8 | PEKIEEANKGEN | epitope |
| HIVNEF_seq_9 | KRQDILDLW | NEF P05856 MHC |
| HIVNEF_seq_10 | HTQGYFPDW | Class 1 T-cell epitope |
| HIVNEF_seq_11 | MRRAEPAEL | NEF P05856 MHC- |
| | | Class 2 T-cell epitope |
| HIVNEF_seq_12 | VKHPRYKGTNDTRN | gp125 Q74432 B Cell |
| HIVNEF_seq_13 | LNWIENKTGRGQKQH | epitope |
| HIVNEF_seq_14 | ATLNRTNITFSAEVAELYRLELGDY | |
| HIVNEF_seq_15 | FAPTPEKRYSSDHGRP | |
| HIVNEF_seq_16 | FRYCAPPGY | gp125 Q74432 MHC |
| | | Class 1 T-cell epitope |
| HIVNEF_seq_17 | WFGFNGTRA | gp125 Q74432 MHC |
| | | Class 2 T-cell epitope |
| HIVNEF_seq_18 | QGGLEGMYYSERRH | NEF- P15829 B-cell |
| HIVNEF_seq_19 | PAATREEEE | epitope |
| HIVNEF_seq_20 | EEFGYQSGL | NEF-P15829 MHC |
| HIVNEF_seq_21 | REEEETHCL | Class-1 T-cell epitope |
| HIVNEF_seq_22 | WKARLKARG | NEF-P15829 MHC |
| | | Class-2 T-cell epitope |
| HIVNEF_seq_23 | GAATAAACTAGTATTCTTCTGGTCCCCA | The 5′-untranslated |
| | CAGACTCAGAGAGAACCCGCCACC | region |
| HIVNEF_seq_24 | ATGTTCGTGTTCCTGGTGCTGCTGCCTC | Signal peptide |
| | TGGTGTCCAGCCAGTGTGTG | |
| HIVNEF_seq_25 | AAACAGAGCAGCGGCGGCGAUCCGGAA | Codon-optimized |
| | AUUGGCGGCGGCGGCAGCGGCGGCGGC | sequence (ORF): U was |
| | GGCAGCGGCGGCGGCGGCAGCGGCGGC | replaced with T for the |
| | GGCGGCAGCACCUGGUUUAACAGCAC | Sequence listing .xml |
| | CUGGAGCACCGAAGGCAGCAACAACA | file. |
| | CCGAAGGCAGCGAUGGCGGCGGCGGC | |
| | AGCGGCGGCGGCGGCAGCGGCGGCGG | |
| | CGGCAGCGGCGGCGGCGGCAGCCUGG | |
| | GCGUGGCGCCGACCAAAGCGAAACGC | |
| | CGCGUGGUGCAGCGCGAAAAAGGCGG | |
| | CGGCGGCAGCGGCGGCGGCGGCAGCG | |
| | GCGGCGGCGGCAGCGGCGGCGGCGGC | |
| | AGCCGCGUGAAAGAAAAAUAUCAGCA | |
| | UCUGUGGGGCGGCGGCGGCAGCGGCG | |
| | GCGGCGGCAGCGGCGGCGGCGGCAGC | |
| | GGCGGCGGCGGCAGCUGGAACGCGAC | |
| | CCUGAAACAGAUUGCGAGCAAACUGC | |
| | GCGAACAGGGCGGCGGCGGCAGCGGC | |
| | GGCGGCGGCAGCGGCGGCGGCGGCAG | |
| | CGGCGGCGGCGGCAGCAAACUGGAUA | |
| | UUAUUCCGAUUGAUAACGAUACCACC | |
| | AGCUAUACCGGCGGCGGCGGCAGCGG | |
| | CGGCGGCGGCAGCGGCGGCGGCGGCAG | |
| | CGGCGGCGGCGGCAGCGGCUAUUUUCC | |
| | GGAUUGGCAGAACUAUACCCCGGGCCC | |
| | GGGCAUUCGCUAUGGCGGCGGCGGCAG | |
| | CGGCGGCGGCGGCAGCGGCGGCGGCGG | |
| | CAGCGGCGGCGGCGGCAGCCCGGAAAA | |
| | AAUUGAAGAAGCGAACAAAGGCGAAAA | |
| | CGGCGGCGGCGGCAGCGGCGGCGGCGG | |
| | CAGCGGCGGCGGCGGCAGCGGCGGCGG | |
| | CGGCAGCAAACGCCAGGAUAUUCUGGA | |
| | UCUGUGGGGCGGCGGCGGCAGCGGCGG | |
| | CGGCGGCAGCGGCGGCGGCGGCAGCGG | |
| | CGGCGGCGGCAGCCAUACCCAGGGCUA | |
| | UUUUCCGGAUUGGGGCGGCGGCGGCAG | |
| | CGGCGGCGGCGGCAGCGGCGGCGGCGG | |
| | CAGCGGCGGCGGCGGCAGCAUGCGCCG | |
| | CGCGGAACCGGCGGAACUGGGCGGCGG | |
| | CGGCAGCGGCGGCGGCGGCAGCGGCGG | |
| | CGGCGGCAGCGGCGGCGGCGGCAGCGU | |
| | GAAACAUCCGCGCUAUAAAGGCACCAA | |
| | CGAUACCCGCAACGGCGGCGGCGGCAG | |
| | CGGCGGCGGCGGCAGCGGCGGCGGCGG | |
| | CAGCGGCGGCGGCGGCAGCCUGAACUG | |
| | GAUUGAAAACAAAACCGGCCGCGGCCA | |
| | GAAACAGCAUGGCGGCGGCGGCAGCGG | |
| | CGGCGGCGGCAGCGGCGGCGGCGGCAG | |
| | CGGCGGCGGCGGCAGCGCGACCCUGAA | |
| | CCGCACCAACAUUACCUUUAGCGCGG | |
| | AAGUGGCGGAACUGUAUCGCCUGGAA | |
| | CUGGGCGAUUAUGGCGGCGGCGGCAG | |
| | CGGCGGCGGCGGCAGCGGCGGCGGCG | |
| | GCAGCGGCGGCGGCGGCAGCUUUGCG | |
| | CCGACCCCGGAAAAACGCUAUAGCAG | |
| | CGAUCAUGGCCGCCCGGGCGGCGGCG | |
| | GCAGCGGCGGCGGCGGCAGCGGCGGC | |
| | GGCGGCAGCGGCGGCGGCGGCAGCUU | |
| | UCGCUAUUGCGCGCCGCCGGGCUAUG | |
| | GCGGCGGCGGCAGCGGCGGCGGCGGC | |
| | AGCGGCGGCGGCGGCAGCGGCGGCGG | |
| | CGGCAGCUGGUUUGGCUUUAACGGCA | |
| | CCCGCGCGGGCGGCGGCGGCAGCGGC | |
| | GGCGGCGGCAGCGGCGGCGGCGGCAG | |
| | CGGCGGCGGCGGCAGCCAGGGCGGCC | |
| | UGGAAGGCAUGUAUUAUAGCGAACGC | |
| | CGCCAUGGCGGCGGCGGCAGCGGCGG | |
| | CGGCGGCAGCGGCGGCGGCGGCAGCG | |
| | GCGGCGGCGGCAGCCCGGCGGCGACCC | |
| | GCGAAGAAGAAGAAGGCGGCGGCGGC | |
| | AGCGGCGGCGGCGGCAGCGGCGGCGG | |
| | CGGCAGCGGCGGCGGCGGCAGCGAAG | |
| | AAUUUGGCUAUCAGAGCGGCCUGGGC | |
| | GGCGGCGGCAGCGGCGGCGGCGGCAG | |
| | CGGCGGCGGCGGCAGCGGCGGCGGCG | |
| | GCAGCCGCGAAGAAGAAGAAACCCAU | |
| | UGCCUGGGCGGCGGCGGCAGCGGCGG | |
| | CGGCGGCAGCGGCGGCGGCGGCAGCG | |
| | GCGGCGGCGGCAGCUGGAAAGCGCGC | |
| | CUGAAAGCGCGCGGC | |
| HIVNEF_seq_26 | GCTAGCTGCCCCTTTCCCGTCCTGGGTACCC | 3′-UTR |
| | CGAGTCTCCCCCGACCTCGGGTCCCAGGTAT | |
| | GCTCCCACCTCCACCTGCCCCACTCACCACCT | |
| | CTGCTAGTTCCAGACACCTCCCAAGCACGCA | |
| | GCAATGCAGCTCAAAACGCTTAGCCTAGCCA | |
| | CACCCCCACGGGAAACAGCAGTGATTAACCT | |
| | TTAGCAATAAACGAAAGTTTAACTAAGCTAT | |
| | ACTAACCCCAGGGTTGGTCAATTTCGTGCCA | |
| | GCCACACCCTGGAGCTAGC | |
| HIVNEF_seq_27 | AAAAAAAAAAAAAAAAAAAAAAAAAAAAA | Poly(A) sequence |
| | AGCATATGACTAAAAAAAAAAAAAAAAAAA | |
| | AAAAAAAAAAAAAAAAAAAAAAAAAAAAAA | |
| | AAAAAAAAAAAAAAAAAAAAA | |
| HIVNEF_seq_28 | KQSSGGDPEIGGGGSGGGGSGGGGSGGGGSTW | Expressed polypeptide |
| | FNSTWSTEGSNNTEGSDGGGGSGGGGSGGGGS | chain (1279) |
| | GGGGSLGVAPTKAKRRVVQREKGGGGSGGGG | |
| | SGGGGSGGGGSRVKEKYQHLWGGGGSGGGGS | |
| | GGGGSGGGGSWNATLKQIASKLREQGGGGSGG | |
| | GGSGGGGSGGGGSKLDIIPIDNDTTSYTGGGGSG | |
| | GGGSGGGGSGGGGSGYFPDWQNYTPGPGIRYG | |
| | GGGSGGGGSGGGGSGGGGSPEKIEEANKGENG | |
| | GGGSGGGGSGGGGSGGGGSKRQDILDLWGGG | |
| | GSGGGGSGGGGSGGGGSHTQGYFPDWGGGGS | |
| | GGGGSGGGGSGGGGSMRRAEPAELGGGGSGG | |
| | GGSGGGGSGGGGSVKHPRYKGTNDTRNGGGG | |
| | SGGGGSGGGGSGGGGSLNWIENKTGRGQKQH | |
| | GGGGSGGGGSGGGGSGGGGSATLNRTNITESA | |
| | EVAELYRLELGDYGGGGSGGGGSGGGGSGGG | |
| | GSFAPTPEKRYSSDHGRPGGGGSGGGGSGGGG | |
| | SGGGGSFRYCAPPGYGGGGSGGGGSGGGGSG | |
| | GGGSWFGFNGTRAGGGGSGGGGSGGGGSGG | |
| | GGSQGGLEGMYYSERRHGGGGSGGGGSGGGG | |
| | SGGGGSPAATREEEEGGGGSGGGGSGGGGSG | |
| | GGGSEEFGYQSGLGGGGSGGGGSGGGGSGG | |
| | GGSREEEETHCLGGGGSGGGGSGGGGSGGG | |
| | GSWKARLKARG | |
| HIVNEF_seq_29 | AAACAGAGCAGCGGCGGCGATCCGGAAAT | Complete mRNA |
| | TGGCGGCGGCGGCAGCGGCGGCGGCGGCA | Sequence |
| | GCGGCGGCGGCGGCAGCGGCGGCGGCGGC | |
| | AGCACCTGGTTTAACAGCACCTGGAGCACC | |
| | GAAGGCAGCAACAACACCGAAGGCAGCGA | |
| | TGGCGGCGGCGGCAGCGGCGGCGGCGGCA | |
| | GCGGCGGCGGCGGCAGCGGCGGCGGCGGC | |
| | AGCCTGGGCGTGGCGCCGACCAAAGCGA | |
| | AACGCCGCGTGGTGCAGCGCGAAAAAGG | |
| | CGGCGGCGGCAGCGGCGGCGGCGGCAGC | |
| | GGCGGCGGCGGCAGCGGCGGCGGCGGCA | |
| | GCCGCGTGAAAGAAAAATATCAGCATCTG | |
| | TGGGGCGGCGGCGGCAGCGGCGGCGGCGG | |
| | CAGCGGCGGCGGCGGCAGCGGCGGCGGCG | |
| | GCAGCTGGAACGCGACCCTGAAACAGAT | |
| | TGCGAGCAAACTGCGCGAACAGGGCGGC | |
| | GGCGGCAGCGGCGGCGGCGGCAGCGGCG | |
| | GCGGCGGCAGCGGCGGCGGCGGCAGCAA | |
| | ACTGGATATTATTCCGATTGATAACGATA | |
| | CCACCAGCTATACCGGCGGCGGCGGCAG | |
| | CGGCGGCGGCGGCAGCGGCGGCGGCGGC | |
| | AGCGGCGGCGGCGGCAGCGGCTATTTTCC | |
| | GGATTGGCAGAACTATACCCCGGGCCCGG | |
| | GCATTCGCTATGGCGGCGGCGGCAGCGGC | |
| | GGCGGCGGCAGCGGCGGCGGCGGCAGCG | |
| | GCGGCGGCGGCAGCCCGGAAAAAATTGA | |
| | AGAAGCGAACAAAGGCGAAAACGGCGG | |
| | CGGCGGCAGCGGCGGCGGCGGCAGCGGC | |
| | GGCGGCGGCAGCGGCGGCGGCGGCAGCA | |
| | AACGCCAGGATATTCTGGATCTGTGGGGC | |
| | GGCGGCGGCAGCGGCGGCGGCGGCAGCG | |
| | GCGGCGGCGGCAGCGGCGGCGGCGGCAG | |
| | CCATACCCAGGGCTATTTTCCGGATTGGGG | |
| | CGGCGGCGGCAGCGGCGGCGGCGGCAGCG | |
| | GCGGCGGCGGCAGCGGCGGCGGCGGCAGC | |
| | ATGCGCCGCGCGGAACCGGCGGAACTGGG | |
| | CGGCGGCGGCAGCGGCGGCGGCGGCAGCG | |
| | GCGGCGGCGGCAGCGGCGGCGGCGGCAGC | |
| | GTGAAACATCCGCGCTATAAAGGCACCAAC | |
| | GATACCCGCAACGGCGGCGGCGGCAGCGG | |
| | CGGCGGCGGCAGCGGCGGCGGCGGCAGCG | |
| | GCGGCGGCGGCAGCCTGAACTGGATTGAA | |
| | AACAAAACCGGCCGCGGCCAGAAACAGCA | |
| | TGGCGGCGGCGGCAGCGGCGGCGGCGGCA | |
| | GCGGCGGCGGCGGCAGCGGCGGCGGCGGC | |
| | AGCGCGACCCTGAACCGCACCAACATTACC | |
| | TTTAGCGCGGAAGTGGCGGAACTGTATCGC | |
| | CTGGAACTGGGCGATTATGGCGGCGGCGGC | |
| | AGCGGCGGCGGCGGCAGCGGCGGCGGCGG | |
| | CAGCGGCGGCGGCGGCAGCTTTGCGCCGAC | |
| | CCCGGAAAAACGCTATAGCAGCGATCATG | |
| | GCCGCCCGGGCGGCGGCGGCAGCGGCGGC | |
| | GGCGGCAGCGGCGGCGGCGGCAGCGGCGG | |
| | CGGCGGCAGCTTTCGCTATTGCGCGCCGC | |
| | CGGGCTATGGCGGCGGCGGCAGCGGCGG | |
| | CGGCGGCAGCGGCGGCGGCGGCAGCGGC | |
| | GGCGGCGGCAGCTGGTTTGGCTTTAACGG | |
| | CACCCGCGCGGGCGGCGGCGGCAGCGGC | |
| | GGCGGCGGCAGCGGCGGCGGCGGCAGCG | |
| | GCGGCGGCGGCAGCCAGGGCGGCCTGGA | |
| | AGGCATGTATTATAGCGAACGCCGCCATG | |
| | GCGGCGGCGGCAGCGGCGGCGGCGGCAG | |
| | CGGCGGCGGCGGCAGCGGCGGCGGCGGC | |
| | AGCCCGGCGGCGACCCGCGAAGAAGAAG | |
| | AAGGCGGCGGCGGCAGCGGCGGCGGCGG | |
| | CAGCGGCGGCGGCGGCAGCGGCGGCGGC | |
| | GGCAGCGAAGAATTTGGCTATCAGAGCG | |
| | GCCTGGGCGGCGGCGGCAGCGGCGGCGG | |
| | CGGCAGCGGCGGCGGCGGCAGCGGCGGC | |
| | GGCGGCAGCCGCGAAGAAGAAGAAACCC | |
| | ATTGCCTGGGCGGCGGCGGCAGCGGCGG | |
| | CGGCGGCAGCGGCGGCGGCGGCAGCGGC | |
| | GGCGGCGGCAGCTGGAAAGCGCGCCTGA | |
| | AAGCGCGCGGC | |

Messenger RNA (mRNA)

mRNA molecules are designed to encode for specific antigens of HIV. The mRNA is structurally composed of a S′UTR, a signal peptide for efficient translation, the open reading frame that encodes the antigen, a 3′UTR for stability, and a polyA tail. This structure ensures efficient translation and antigen presentation for an immune response. Using pseudouridine in the mRNA is a modification to avoid innate immune sensing and enhance translation efficiency. An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs. However, unlike the ORF, those elements need not necessarily be present in a vaccine of the present disclosure.

The mRNA vaccine platform is safe, does not infect the body or integrate into the genome, and does not carry the risk of infection or mutation. Its immune effect is better and better stabilized by various modifications and encapsulation, yielding strong humoral and cellular immune responses. It is also most convenient to produce at a low cost, making the mRNA vaccine a significant humanitarian assistance. The mRNA vaccine can be produced rapidly on a large scale by an in vitro transcription technology, and compared with the traditional vaccine with a production period of 5-6 months or longer, the production preparation of the mRNA vaccine sample can be completed within dozens of days. The outburst epidemic situation can be dealt with more efficiently.

After the mRNA vaccine containing the mRNA molecule is used for immunization, an organism can simultaneously generate humoral and cellular immune responses aiming at a plurality of antigens in an induced mode. Therefore, a better immune effect is achieved. Thus, the claimed invention comprises a mixture of multiple mRNA molecules, each capable of expressing one antigenic protein, two for HIV, and multiple for Nef protein immunity generation.

The nucleoside modifications within the mRNA are crucial because they help to evade the host's innate immune responses, which can often degrade mRNA before it achieves its purpose. These modifications can enhance the translational capacity and stability of the mRNA, leading to higher and more prolonged protein expression of the vaccine antigen within the body. As a result, these vaccines can induce robust and sustained immune responses, which are critical for preventive and therapeutic vaccine strategies.

TABLE 2
Sequences of mRNA structure

| Element | Description |
|---|---|
| Cap (2) | A modified 5′-cap1 structure (m7G + m3′-5′-ppp- |
| | 5′-Am): GA |
| 5′-UTR | The 5′-untranslated region derived from human |
| (52) | alpha-globin RNA with an optimized Kozak sequence. |
| | [SEQUENCE NO. 23] |
| Signal | S glycoprotein signal peptide (extended leader |
| peptide | sequence) guides translocation of the nascent |
| (48) | polypeptide chain into the endoplasmic reticulum: |
| | [SEQUENCE NO. 24] |
| Coding | Codon-optimized sequence (ORF): 25 |
| region | [SEQUENCE 25] |
| (3837) | |
| 3′-UTR | The 3′ untranslated region comprises two sequence |
| (268) | elements derived from the amino-terminal enhancer |
| | of split (AES) mRNA and the mitochondrial encoded 12S |
| | ribosomal RNA to confer RNA stability and high total |
| | protein expression: |
| | [SEQUENCE NO. 26] |
| poly(A) | A 110-nucleotide poly(A)-tail consisting of a stretch |
| (110) | of 30 adenosine residues, followed by a 10-nucleotide |
| | linker sequence and another 70 adenosine residues: |
| | [SEQUENCE NO. 27] |

### Lipid Nanoparticle (LNP)

The LNP delivery system is an essential component of the invention, allowing for the encapsulation and delivery of the mRNA into human cells. The LNPs are designed to include an ionizable cationic lipid for effective delivery, a non-cationic lipid for structural stability, and a PEGylated lipid to extend circulation time in the bloodstream. Lyophilization of the LNP is included to enhance stability and shelf-life, making the vaccine suitable for distribution and storage.

In some respects, the present disclosure provides compositions of inducing in a human subject an immune response to HIV, the compositions comprising administering to the subject a lipid nanoparticle comprising a mRNA encoding epitopes of HIV gp160 proteins P03377 ENV_HVIBR, andC6GOE7_9HIV1.

Lyophilization of LNP formulation extends the storage life of the product. It allows storage at higher temperatures, making it an essential consideration for distributing the HIV vaccine that is direly needed in many countries where storage conditions are non-compatible with non-lyophilized products.

Endosomes are composed of a lipid bilayer barrier that has served to prevent invading nucleic acids from entering the cell for over a billion years. For more than 50 years, the endosomal lipid bilayer has also served as a substantial barrier to delivering precision genetic medicines. As endosomes mature, their pH drops from ˜7.2 to 6 or less when fused to lysosomes. Fortunately, ionizable lipids are designed with a pKa ˜6.2, so as the endosome matures, they go from being neutral to becoming protonated and positively charged. This results in a phase transformation of the ionizable lipid from a cone shape into a hexagonal Hu structure. This results in a localized disruption to the endosomal lipid bilayer followed by endosomal escape of the precision genetic medicine into the cell's cytoplasm. While ionizable LNPs have a built-in mechanism to address the endosomal escape problem, only ˜1% of endocytosed ionizable LNPs escape the endosome. Thus, further enhancing endosomal escape above the 1% ceiling in a non-toxic manner remains a significant problem to be solved. The present invention further includes the delivery of LNP through endosomes.

### EMBODIMENTS

In the first embodiment, the present invention uses multiple mRNA molecules or a single molecule capable of expressing various antigens to provide a vaccine to prevent or treat HIV.

In the second embodiment, the present invention provides mRNA molecules, each expressing selected T-cell and B-cell epitopes of HIV gp160 protein (UniProt: P03377 ENV_HVIBR and its Nef protein HIV-1 NEF P05856 and HIV-2 gp125 Q74432 and its Nef protein HIV-2 NEF-P15829.

In a third embodiment, the present invention provides a therapeutic mRNA molecule comprising a 5′UTR element, a signal peptide element, an open reading frame corresponding to the expressed proteins, a 3′ UTR element, and a polyA tail element linked together in that order.

In a fourth embodiment, the uracil, cytosine, or adenine nucleotides of the therapeutic mRNA molecule contain a modifying group that includes at least one of pseudouridine, N1-methyl pseudouridine, N1-ethylpseudouridine, 5-methylcytosine, 2-thiouridine, 5-methoxyuridine, or N1-methyladenosine. RNA sequences are modified by replacing uridine (U) with pseudouridine (Y).

In a fifth embodiment, the present invention claims an LNP formulation that comprises the steps of mixing D-Lin-MC3-DMA, DSPC, cholesterol, and DMG-PEG 2000 in an absolute ethanol solution, adding the mixture into a citrate buffer solution, and extruding the mixture by a liposome extruder to obtain the liposome nanoparticle.

1. A coding mRNA vaccine to elicit an adaptive immune response against the human immunodeficiency virus (HIV) comprising: a) at least one 5′-cap structure; b) at least one heterologous 5′ untranslated region (5′-UTR);c) at least one heterologous 3′ untranslated region (3′-UTR);d) at least one signal peptide; e) at least one poly(A) tail;e) at least one coding open reading frame (ORF) to express B-cell and T-cell epitopes of HIV-1 proteins gp160 (P03377) and its Nef protein (P05856), gp125 (Q74432) and its Nef protein (P15829), in a single or multiple amino acid chain.

2. The coding mRNA vaccine of claim 1, wherein the coding mRNA is a self-replicating RNA, a circular RNA, or a replicon RNA.

3. The coding mRNA vaccine of claim 1, wherein the B-cell and T-cell epitope selected from a group of epitopes comprising HIVNEF_seq_1 to HIVNEF_seq_22, or a combination thereof.

4. The coding mRNA vaccine of claim 1, wherein the epitopes are linked by a linker group comprising Alanine, Asparagine, Glutamic Acid, Glycine, Leucine, Lysine, Phenylalanine, Proline, Serine, and Threonine, or a combination thereof, preferably Glycine-Glycine-Glycine-Glycine-Serine.

5. The coding mRNA vaccine of claim 1, wherein the 5′UTR Cap comprises a m7G+m3′-5′-ppp-5′-Am cap GP.

6. The coding mRNA vaccine of claim 1, wherein the heterologous 5′ untranslated region 5′-UTR comprises HIVNEF_seq_23.

7. The coding mRNA vaccine of claim 1, wherein the heterologous signal peptide comprises HIVNEF_seq_24.

8. The coding mRNA vaccine of claim 1, wherein the ORF comprises HIVNEF_seq_25.

9. The coding mRNA vaccine of claim 1, wherein the heterologous 3′ untranslated region 3′-UTR comprises HIVNEF_seq_26.

10. The coding mRNA vaccine of claim 1, wherein the mRNA comprises a poly(A) sequence, comprising 30 to 150 adenosine nucleotides, preferably the HIVNEF_seq_27.

11. The coding mRNA vaccine of claim 1, wherein the vaccine expresses a polypeptide comprising HIVNEF_seq_28.

12. The coding mRNA vaccine of claim1, wherein the entire sequence of the mRNA vaccine comprises HIVNEF_seq_29.

13. The coding mRNA vaccine of claim 1, wherein RNA sequences are codon-optimized by replacing uridine (U) either wholly or partially by pseudouridine and N1-methyl pseudouridine (T), or a combination thereof.

14. The composition of claim 1, wherein the mRNA molecules are enclosed in lipid nanoparticles further comprising an ionizable cationic lipid, a non-cationic lipid, and a PEG-modified lipid.

15. The coding mRNA vaccine of claim 14, wherein the coding mRNA is complexed or associated with or at least partially complexed or partially associated with one or more cationic or polycationic compounds, preferably cationic or polycationic polymer, cationic or polycationic polysaccharide, cationic or polycationic lipid, cationic or polycationic protein, cationic or polycationic peptide, or any combinations thereof.

16. The coding mRNA vaccine of claim 14, wherein the coding mRNA is complexed or associated with one or more lipids, thereby forming liposomes, lipid nanoparticles (LNP), lipoplexes, and nanoliposomes, presented with or without lyophilization.

17. The coding mRNA vaccine of claim 16, wherein the LNP essentially consists of (i) at least one cationic lipid, (ii) at least one neutral lipid, (iii) at least one steroid or steroid analog;
and (iv) at least once a PEG-lipid.

18. The coding mRNA vaccine of claim 16, wherein the lipid nanoparticles are lyophilized.

19. The coding mRNA vaccine of claim 16, wherein the lipid nanoparticle formulation comprises the steps of mixing D-Lin-MC3-DMA, DSPC, cholesterol, and DMG-PEG 2000 in an absolute ethanol solution, adding the mixture into a citrate buffer solution, and extruding the mixture by a liposome extruder to obtain the liposome nanoparticle.

20. The coding mRNA vaccine of claim 19, wherein the LNP is delivered packaged in an endosome.

21. The coding mRNA vaccine of claim 1, wherein the mRNA vaccine is used to treat or prevent infection from HIV.


SEQUENCE LISTING (8 sequences)

SEQ ID NO: 1 - protein, 111 aa, synthetic construct
MEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVL

SEQ ID NO: 2 - protein, 120 aa, synthetic construct
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 3 - RNA, 52 nt, synthetic construct
  source 1..52: mol_type = other RNA
gaataaactagtattcttctggtccccacagactcagagagaacccgccacc

SEQ ID NO: 4 - RNA, 48 nt, Synthetic construct
  source 1..48: mol_type = mRNA
atgttcgtgttcctggtgctgctgcctctggtgtccagccagtgtgtg

SEQ ID NO: 5 - RNA, 750 nt, Synthetic construct
  source 1..750: mol_type = mRNA
gaaattgtgatgacccagagcccgagcaccctgagcgcgagcgtgggcgatcgcgtgattattacctgccaggcgagcgaaattattcatagctggctggcgtggtatcagcagaaaccgggcaaagcgccgaaactgctgatttatctggcgagcaccctggcgagcggcgtgccgagccgctttagcggcagcggcagcggcgcggaatttaccctgaccattagcagcctgcagccggatgattttgcgacctattattgccagaacgtgtatctggcgagcaccaacggcgcgaactttggccagggcaccaaactgaccgtgctgggcggcggcggcagcggcggcggcggcagcggcggcggcggcagcggcggcggcggcagcgaagtgcagctggtggaaagcggcggcggcctggtgcagccgggcggcagcctgcgcctgagctgcaccgcgagcggctttagcctgaccgattattattatatgacctgggtgcgccaggcgccgggcaaaggcctggaatgggtgggctttattgatccggatgatgatccgtattatgcgacctgggcgaaaggccgctttaccattagccgcgataacagcaaaaacaccctgtatctgcagatgaacagcctgcgcgcggaagataccgcggtgtattattgcgcgggcggcgatcataacagcggctggggcctggatatttggggccagggcaccctggtgaccgtgagcagc

SEQ ID NO: 6 - RNA, 268 nt, Synthetic construct
  source 1..268: mol_type = mRNA
gctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 7 - RNA, 110 nt, Synthetic construct
  source 1..110: mol_type = mRNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 8 - protein, 251 aa, synthetic construct
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS